(19) United States
(12) Patent Application Publication
(10) Pub. No.: US 2008/0145325 A1
(43) Pub. Date: Jun. 19, 2008

(54) Nail varnish comprising at least one resin

(76) Inventors: Lizabeth-Anne Coffey-Dawe, Aulnay Sous Bois (FR); Laurence Guerchet, Chertty-Loive (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/898,532

(22) Filed: Sep. 13, 2007

Related U.S. Application Data
(60) Provisional application No. 60/845,526, filed on Sep. 19, 2006.

(30) Foreign Application Priority Data
Sep. 13, 2006 (FR) 0653710

(51) Int. Cl. A61K 8/35; A61Q 3/02

(52) U.S. Cl. 424/61

(57) ABSTRACT

The present invention relates to a nail varnish comprising an organic solvent medium, a gelling agent and at least one ketone/aldehyde resin. The present invention also relates to a cosmetic process for making up or for the non-therapeutic care of the nails, comprising the application to the nails of at least one coat of the nail varnish, and also to its use for obtaining a glossy film after deposition on the nail.

This application claims benefit of U.S. Provisional Application No. 60/845,526, filed Sep. 19, 2006, the contents of which are incorporated herein by reference. This application also claims benefit of priority under 35 U.S.C. § 119 to French Patent Application FR 06/53710, filed Sep. 13, 2006, the contents of which are also incorporated herein by reference.

Disclosed herein is a nail varnish composition comprising an organic solvent medium and at least one ketone/aldehyde resin. This composition may be applied to human nails or to false nails.

The nail varnish may be colored or transparent and may be used as a varnish base or base coat, as a nail makeup product, as a finishing composition, also known as a top coat, to be applied over the nail makeup product, and/or as a cosmetic nailcare product.

It is common practice to use thickeners in nail varnish compositions, in order to increase the viscosity of the organic phase of these compositions. Thickening of the organic phase makes it possible, for example, to improve the suspension of the pigments present in this phase and to avoid their sedimentation during storage. Thickening of the organic phase may also facilitate the uptake of the composition from its packaging and the distribution of the composition onto the area to be treated.

It is common practice to use thickeners, for instance, clays such as Bentone, in order to thicken the organic phase of nail varnishes. However, the fact that Bentone is in the form of particles of the size of about one micron causes matting of the film of nail varnish formed and substantial loss of gloss of this film. It is thus desirable to improve the gloss of nail varnish compositions comprising thickeners of this type.

Thus disclosed herein are nail varnish compositions that have improved gloss, and at the same time exhibit good stability over time, and allow the formation of a uniform film on the nails.

The present inventors have discovered that it is possible to increase the gloss of nail varnish compositions by introducing therein at least one specific resin.

Disclosed herein, therefore, is a nail varnish comprising an organic solvent medium, at least one gelling agent, and at least one ketone/aldehyde resin.

Also disclosed herein is a cosmetic process for making up and/or for the non-therapeutic care of the nails, comprising applying to the nails at least one coat of a nail varnish as defined herein.

Further disclosed herein is the use of a nail varnish comprising an organic solvent medium, at least one gelling agent and at least one ketone/aldehyde resin, to obtain a glossy film after deposition on the nail.

Cosmetically Acceptable Medium

The nail varnish according to the present disclosure comprises a cosmetically acceptable medium, i.e., a non-toxic medium that may be applied to human keratin materials, such as the nails.

Organic Solvent Medium

The cosmetically acceptable medium may be chosen, for example, from organic solvent mediums comprising at least one organic solvent or a mixture of organic solvents.

The organic solvent may be chosen, by way of non-limiting example, from:

ketones that are liquid at room temperature, such as methyl ethyl ketone, methyl isobutyl ketone, diisobutyl ketone, isophorone, cyclohexanone, and acetone;

alcohols that are liquid at room temperature, such as ethanol, isopropanol, diacetone alcohol, 2-butoxyethanol, and cyclohexanol;

propylene glycol ethers that are liquid at room temperature, such as propylene glycol monomethyl ether, propylene glycol monomethyl ether acetate, and dipropylene glycol mono-n-butyl ether;

cyclic ethers such as γ-butyrolactone;

short-chain esters, for example, esters comprising from 3 to 8 carbon atoms, such as ethyl acetate, methyl acetate, propyl acetate, isopropyl acetate, n-butyl acetate, isopentyl acetate, methoxypropyl acetate, t-butyl acetate, and butyl lactate;

ethers that are liquid at room temperature, such as diethyl ether, dimethyl ether, and dichlorodiethyl ether;

alkanes that are liquid at room temperature, such as decane, heptane, dodecane, and cyclohexane;

alkyl sulfoxides such as dimethyl sulfoxide;

aldehydes that are liquid at room temperature, such as benzaldehyde and acetaldehyde;

ethyl 3-ethoxypropionate;

carbonates such as propylene carbonate and dimethyl carbonate;

acetals such as methylal; and

mixtures thereof.

In at least one embodiment, the solvent is chosen from short-chain esters comprising from 3 to 8 carbon atoms, such as ethyl acetate, methyl acetate, propyl acetate, isopropyl acetate, n-butyl acetate, isopentyl acetate, methoxypropyl acetate, butyl lactate, and mixtures thereof.

The organic solvent medium may represent from 10% to 95% by weight, such as from 15% to 80% by weight, or from 20% to 70% by weight relative to the total weight of the composition.

Aqueous Medium

The cosmetically acceptable medium of the composition according to the present disclosure may also be chosen from aqueous mediums.

The aqueous medium may be present in the composition in an amount ranging from 5% to 95% by weight, for example, from 50% to 70% by weight relative to the total weight of the composition.

According to at least one embodiment, the composition comprises water in an amount of less than 10% by weight, for example, less than 5% by weight of water, relative to the total weight of the composition.

Ketone/Aldehyde Resin

The nail varnish composition according to the present disclosure comprises at least one ketone/aldehyde resin.

Such ketone/aldehyde resins may be chosen, for example, from products of polycondensation (comprising a dehydration step) of mixtures of at least one ketone and of at least one aldehyde.

These resins may be prepared according to the processes described, for instance, in U.S. Patent Application Publication Nos. 2005/0080222 and 2005/0124716, which are incorporated herein by reference in their entireties.

The at least one ketone that may be used for the preparation of the resins according to the present disclosure may be chosen from aliphatic ketones, such as those comprising from 1 to 20, for example, from 1 to 10 carbon atoms, and/or cycloaliphatic ketones, such as those comprising from 5 to 20, for example, from 6 to 12 carbon atoms, and mixtures thereof. Non-limiting examples of such ketones include acetone, acetophenone, methyl ethyl ketone, 2-heptanone, methyl isobutyl ketone, cyclopentanone, cyclododecanone, mixtures of 2,2,4- and 2,4,4-trimethyl cyclohexanone, cyclohexanone, mixtures of cycloheptanone and cyclooctanone, and cyclohexanones substituted with at least one alkyl group, for instance, alkyl groups comprising from 1 to 8 carbon atoms. Examples of cyclohexanones substituted with at least one alkyl group include, but are not limited to, 4-tert-amylcyclohexanone, 2-sec-butylcyclohexanone, 4-tert-butylcyclohexanone, 2-methylcyclohexanone, 3,3,5-trimethylcyclohexanone, and mixtures thereof.

According to one embodiment, the at least one ketone used to prepare the ketone/aldehyde resins of the present disclosure may be cycloaliphatic ketones, such as acetophenone, cyclohexanone, 4-tert-butylcyclohexanone and 3,3,5-trimethylcyclohexanone, and mixtures thereof.

According to another embodiment, the at least one ketone is acetophenone.

The at least one aldehyde that may be used for the preparation of the ketone/aldehyde resins according to the present disclosure may be chosen from linear aldehydes, such as those comprising from 1 to 20, for example, from 1 to 15, carbon atoms; branched aldehydes, such as those comprising from 5 to 20, for example, from 6 to 12 carbon atoms, and mixtures thereof, for instance, formaldehyde, acetaldehyde, n-butyraldehyde, iso-butyraldehyde, valeraldehyde, dodecanal, and mixtures thereof.

According to one embodiment, the at least one aldehyde used for the synthesis of the ketone/aldehyde resins of the present disclosure is chosen from formaldehyde and mixtures of formaldehyde with other aldehydes.

According to another embodiment, the at least one aldehyde is formaldehyde.

In yet another embodiment, the aldehydes may be in the form of an aqueous or alcoholic solution (for example, as a solution in methanol or butanol), for instance, present in an aqueous or alcoholic solution in a concentration ranging from 20% to 40% by weight.

According to a further embodiment, the ketone/aldehyde resins are acetophenone/formaldehyde resins.

In at least one embodiment, the ketone/aldehyde resins may be introduced into the nail varnish compositions according to the present disclosure in a hydrogenated form or in a modified form.

Hydrogenated Ketone/Aldehyde Resins

According to one embodiment, the ketone/aldehyde-resins may be hydrogenated in the presence of a suitable catalyst and at a pressure ranging up to 300 bar. During this hydrogenation step, the carbonyl group of the ketone/aldehyde resin is converted into a hydroxyl group.

According to at least one embodiment, the ketone/aldehyde resins may be hydrogenated acetophenone/formaldehyde resins obtained by the following reaction scheme:

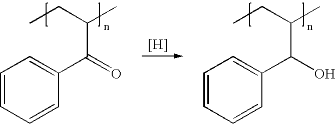

Modified Ketone/Aldehyde Resins

The modified ketone/aldehyde resins may also be obtained by reacting at least one hydrogenated ketone/aldehyde resin described herein and at least one compound capable of reacting with the hydrogenated ketone/aldehyde resin.

By way of non-limiting example, the compound capable of reacting with the hydrogenated ketone/aldehyde resin may be chosen from maleic anhydrides, (meth)acrylic acid derivatives, and isocyanates, such as diisocyanates and polyisocyanates comprising more than two isocyanate groups per molecule.

The diisocyanates that may be used to modify the hydrogenated ketone/aldehyde resin may include, but are not limited to, linear and branched C₁-C₁₂, for example, C₁-C₆, alkyl diisocyanates, C₄-C₂₀ cycloalkyl diisocyanates, and C₆-C₂₀ aryl diisocyanates. In at least one embodiment, the diisocyanates may be chosen from hexamethylene diisocyanate (HDI), cyclohexyl diisocyanate, methyl cyclohexyl diisocyanate, ethyl cyclohexyl diisocyanate, propyl cyclohexyl diisocyanate, methyl diethyl cyclohexyl diisocyanate, phenyl diisocyanate, tolylene diisocyanate, bis(isocyanatophenyl)methane, propane diisocyanate, butane diisocyanate, pentane diisocyanate, hexane diisocyanate, heptane diisocyanate, octane diisocyanate, nonane diisocyanate, isophorone diisocyanate (IPDI), bis(isocyanatomethyl cyclohexyl)methane (H₁₂MDI), isocyanato methyl methyl cyclohexyl isocyanate, and mixtures thereof.

According to one embodiment, the compound used to modify the hydrogenated ketone/aldehyde resin may be a diisocyanate such as isophorone diisocyanate (IPDI).

According to another embodiment, the ketone/aldehyde resin used in the compositions of the present disclosure is a hydrogenated acetophenone/formaldehyde resin modified with isophorone diisocyanate (IPDI), obtained according to the following reaction scheme:

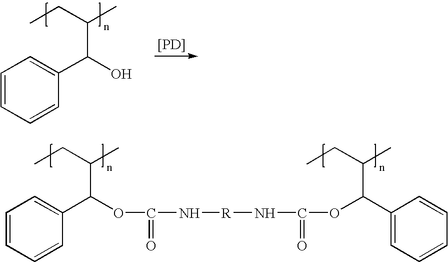

The at least one ketone/aldehyde resin that may be used in the compositions according to the present disclosure may also be chosen, by way of non-limiting example, from the resins sold under the name Synthetic Resin SK and Synthetic Resin 1201 by the company Degussa.

The at least one ketone/aldehyde resin may be present in the compositions according to the present disclosure in an amount ranging from 0.01% to 20% by weight, for example, from 0.05% to 15% by weight, or from 0.1% to 8% by weight of active material relative to the total weight of the composition.

Film-Forming Polymers

The composition according to the present disclosure may further comprise at least one film-forming polymer.

As used herein, the term “film-forming polymer” means a polymer that is capable, by itself or in the presence of an auxiliary film-forming agent, of forming a continuous film on a support, for example, on keratin materials.

Film-forming polymers that may be used in the composition of the present disclosure include, but are not limited to, free-radical synthetic polymers, polycondensate synthetic polymers, polymers of natural origin, and mixtures thereof.

The film-forming polymer may be chosen, by way of non-limiting example, from cellulose-based polymers such as nitrocellulose, cellulose acetate, cellulose acetobutyrate, cellulose acetopropionate, and ethylcellulose; polyurethanes; acrylic polymers; vinyl polymers; polyvinyl butyrals; alkyd resins; and resins derived from aldehyde condensation products such as arylsulfonamide-formaldehyde resins, for instance, toluenesulfonamide-formaldehyde resin, arylsulfonamide-epoxy resins; and ethyltosylamide resins.

Examples of commercially-available film-forming polymers that may be used include, but are not limited to, nitrocellulose RS ⅛ sec.; RS ¼ sec.; RS ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; and SS 5 sec.; sold by the company Hercules; the toluenesulfonamide-formaldehyde resins Ketjenflex MS80 available from the company Akzo; Santolite MHP and Santolite MS 80 available from the company Faconnier, Resimpol 80 available from the company Pan Americana, the alkyd resin Beckosol ODE 230-70-E available from the company Dainippon, the acrylic resin Acryloid B66 available from the company Rohm & Haas, and the polyurethane resin Trixene PR 4127 available from the company Baxenden.

According to one embodiment of the present disclosure, the at least one film-forming polymer is a film-forming linear block ethylenic polymer, comprising, for example, at least one first block and at least one second block with different glass transition temperatures (Tg), the first and second blocks being linked together by an intermediate block comprising at least one constituent monomer of the first block and at least one constituent monomer of the second block.

In another embodiment, the first and second blocks of the block polymer are mutually incompatible.

Such polymers are described, for example, in European Patent Application No. 1 411 069 and International Patent Application Publication No. WO-04/028488.

The at least one film-forming polymer may be present in the composition in an amount ranging from 0.1% to 60% by weight, for example, from 2% to 40% by weight, or from 5% to 25% by weight relative to the total weight of the composition.

Auxiliary Film-Forming Agents

The composition of the present disclosure may further comprise at least one auxiliary-film forming agent, for example, to improve the film-forming properties of the nail varnish composition.

Such an auxiliary film-forming agent may be chosen from any compound known to those skilled in the art as being capable of satisfying the desired function, for example, plasticizers and coalescers for the at least one film-forming polymer.

Thus, the composition may also comprise at least one plasticizer and/or at least one coalescer. Non-limiting examples of common plasticizers and coalescers include:

glycols and derivatives thereof, such as diethylene glycol ethyl ether, diethylene glycol methyl ether, diethylene glycol butyl ether, diethylene glycol hexyl ether, ethylene glycol ethyl ether, ethylene glycol butyl ether, and ethylene glycol hexyl ether;

glycol esters;

propylene glycol derivatives, for example, propylene glycol phenyl ether, propylene glycol diacetate, dipropylene glycol ethyl ether, tripropylene glycol methyl ether, diethylene glycol methyl ether, and propylene glycol butyl ether;

acid esters, for example, carboxylic acid esters, such as citrates, phthalates, adipates, carbonates, tartrates, phosphates, and sebacates;

oxyethylenated derivatives, such as oxyethylenated oils, for instance, plant oils such as castor oil; and

mixtures thereof.

The type and amount of auxiliary film-forming agent such as plasticizers and/or coalescers may be chosen by a person skilled in the art on the basis of his or her general knowledge.

For example, the at least one auxiliary film-forming agent may be present in the composition in an amount ranging from 0.01% to 20%, for instance, from 0.5% to 10% by weight relative to the total weight of the composition.

Gelling Agents

The composition may further comprise at least one gelling agent (or thickener).

The at least one gelling agent or thickener may be present in the composition according to the present disclosure in an amount ranging from 0.01% to 15% by weight, for example, from 0.5% to 15%, or from 0.5% to 10% by weight relative to the total weight of the composition.

This gelling agent may be chosen, by way of non-limiting example, from: hydrophobic silicas, such as those described in European Patent No. 0 898 960, and sold, for example, under the references Aerosil R812® by the company Degussa, Cab-O-Sil TS-530®, Cab-O-Sil TS-610®, and Cab-O-Sil TS-720® by the company Cabot, and Aerosil R972® and Aerosil R974® by the company Degussa; organophilic clays such as montmorillonite, modified clays, for instance, bentones, stearalkonium hectorite, and stearalkonium bentonite, and polysaccharide alkyl ethers (such as those in which the alkyl group contains from 1 to 24, for example, from 1 to 10, from 1 to 6, or from 1 to 3 carbon atoms) such as those described in European Patent Application No. 0 898 958.

In one embodiment, the at least one gelling agent comprises at least one organophilic clay.

Organophilic clays are clays modified with a chemical compound chosen, for example, from quaternary amines, tertiary amines, amine acetates, imidazolines, amine soaps, fatty sulfates, alkyl aryl sulfonates, amine oxides, and mixtures thereof.

Organophilic clays that may be mentioned include, but are not limited to, quaternium-18 bentonites such as those sold under the names Bentone 3, Bentone 38, and Bentone 38V by the company Elementis, Tixogel VP by the company United Catalyst, and Claytone 34, Claytone 40 and Claytone XL by the company Southern Clay; stearalkonium bentonites such as those sold under the names Bentone, 27V by the company Elementis, Tixogel LG by the company United Catalyst, and Claytone AF and Claytone APA by the company Southern Clay; and quaternium-18/benzalkonium bentonites such as those sold under the names Claytone HT and Claytone PS by the company Southern Clay.

According to one embodiment, the at least one organophilic clay may be chosen from modified hectorites such as hectorite modified with benzyldimethylammonium stearate.

The at least one organophilic clay may be present in the composition in an amount of greater than or equal to 0.5% by weight, for example, ranging from 0.5% to 10% by weight, or greater than or equal to 0.7% by weight, for example, ranging from 0.7% to 5% by weight, relative to the total weight of the composition.

Dyestuffs and Fillers

The composition according to the present disclosure may further comprise at least one dyestuff chosen from water-soluble dyes and pulverulent dyestuffs, for instance, pigments, nacres, and flakes that are well known to those skilled in the art. The at least one dyestuff may be present in the composition in an amount ranging from 0.01% to 50% by weight, for example, from 0.01% to 30% by weight relative to the weight of the composition.

As used herein, the term “pigments” is understood as meaning white or colored, mineral or organic particles of any form, which are insoluble in the physiological medium and are intended to color the composition.

As used herein, the term “nacres” is understood as meaning iridescent particles of any form, such as those produced by certain mollusks in their shell, or else synthesized.

The pigments may be white or colored, and mineral and/or organic. Examples of mineral pigments include, but are not limited to, optionally surface-treated titanium dioxide, zirconium oxide, cerium oxide, zinc oxide, iron (e.g., black, yellow, and red) oxide, chromium oxide, manganese violet, ultramarine blue, chromium hydrate, ferric blue, and metal powders, for instance, aluminum powder and copper powder.

Non-limiting examples of organic pigments include carbon black, pigments of D&C type and lakes based on cochineal carmine, barium, strontium, calcium, and/or aluminum.

Suitable nacreous pigments may be chosen, for example, from white nacreous pigments such as mica coated with titanium or with bismuth oxychloride, colored nacreous pigments such as titanium mica coated with iron oxides, titanium mica coated with ferric blue or with chromium oxide, titanium mica coated with an organic pigment of the abovementioned type, and nacreous pigments based on bismuth oxychloride.

The water-soluble dyes may include, for example, beetroot juice and methylene blue.

The composition according to the present disclosure may further comprise at least one filler, present in an amount ranging from 0.01% to 50% by weight, for example, from 0.01% to 30% by weight relative to the total weight of the composition. As used herein, the term “fillers” is understood as meaning colorless or white, mineral or synthetic particles of any form, which are insoluble in the medium of the composition irrespective of the temperature at which the composition is manufactured. These fillers may serve to modify the rheology and/or the texture of the composition.

The fillers may be chosen from mineral or organic fillers of any form, such as platelet-shaped, spherical, and oblong, irrespective of the crystallographic form (for example, lamellar, cubic, hexagonal, orthorhombic, etc.). Non-limiting examples of such fillers include talc, mica, silica, kaolin, polyamide (Nylon®) powder (Orgasol® from Atochem), poly-β-alanine powder, polyethylene powder, tetrafluoroethylene polymer (Teflon®) powder, lauroyllysine, starch, boron nitride, hollow polymer microspheres such as polyvinylidene chloride/acrylonitrile microspheres, for instance Expancel® (Nobel Industrie), acrylic acid copolymers (Polytrap® from the company Dow Corning), silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass and ceramic microcapsules, and metal soaps derived from organic carboxylic acids comprising from 8 to 22 carbon atoms, such as from 12 to 18 carbon atoms, for example, zinc stearate, magnesium stearate, lithium stearate, zinc laurate, and magnesium myristate.

Other Additives

The composition may also comprise other ingredients commonly used in cosmetic compositions. Such ingredients may be chosen, for example, from spreading agents, wetting agents, dispersants, antifoams, preserving agents, UV-screening agents, active agents, surfactants, moisturizers, fragrances, neutralizers, stabilizers, and antioxidants.

It is to be understood that a person skilled in the art will take care to select the at least one optional additional compound and/or the amount thereof, such that the advantageous properties of the composition of the present invention are not, or are not substantially, adversely affected by the envisaged addition.

Also disclosed herein is a nail varnish product comprising: i) a container delimiting at least one compartment, the container being closed by means of a closing member, and ii) a composition according to the present disclosure that is placed inside the at least one compartment.

The container may be in any suitable form, for example, bottles, and may be at least partly made of a material such as glass. However, materials other than glass may be used, for instance, thermoplastics such as PP and PE and metals.

The closing member may be coupled to the at least one compartment by screwing the container in the closed position. Alternatively, the coupling between the closing member and the container may take place other than by screwing, for example, by click-fastening.

The container may further be equipped with an applicator that may be in the form of a fine brush consisting of at least one tuft of bristles. Alternatively, the applicator may be in a form other than a fine brush, for example, spatulas and foam tips.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below. Unless otherwise mentioned, the amounts are given as weight percentages relative to the total weight of the composition.

### EXAMPLES

### Example 1

### Nail Varnish

Two nail varnishes having the following compositions (weight %) were prepared:

| | Inventive | Comparative |
|---|---|---|
| | Composition 1 | Composition 1 |
| Nitrocellulose containing 30% | 11.49 | 12.96 |
| isopropyl alcohol (viscosity: | | |
| E22-1/2 S) | | |
| Pure isopropyl alcohol | 3.67 | 3.67 |
| Nitrocellulose containing 30% | 1.18 | 1.44 |
| isopropyl alcohol (Azur E80 | | |
| from Bergerac) | | |
| Hydrogenated ketone/formalde- | 5.00 | 0.00 |
| hyde resin ⁽¹⁾ | | |
| Ethyl tosylamide resin | 2.92 | 2.92 |
| Glycerophthalic alkyd resin | 16.17 | 16.17 |
| esterified with branched fatty | | |
| acids, at 70% in ethyl acetate | | |
| (Beckosol ODE 230 70E from | | |
| Dainippon Ink and Chemicals) | | |
| Hectorite modified with stearyl- | 1.31 | 1.31 |
| benzyl-dimethylammonium | | |
| (Bentone 27 V from Elementis) | | |
| Red lake sold under the name D&C | 0.21 | 0.21 |
| Red 7 CA Lake C 19-011 by the | | |
| company Sun | | |
| Titanium oxide coated with poly- | 0.02 | 0.02 |
| ethylene oxide | | |
| Bordeaux lake sold under the name | 0.56 | 0.56 |
| D&C Red 34 CA Lake C 24-012 by the | | |
| company Sun | | |
| Ethyl acetate | 20.75 | 22.97 |
| Tributyl acetyl citrate | 3.95 | 3.95 |
| Butyl acetate | 32.72 | 33.77 |
| Citric acid monohydrate | 0.05 | 0.05 |

⁽¹⁾ sold under the name Synthetic Resin SK by the company Degussa.

The gloss of each varnish was measured according to the following protocol:

A coat with a wet thickness of 300 μm of the composition whose mean gloss it was sought to evaluate was spread onto a Leneta brand contrast card of reference Form 1A Penopac, using an automatic spreader. The coat covered the white background and the black background of the card. The composition was left to dry for 24 hours on a bench thermostatically maintained at 30° C., and the gloss at 20° and 60° was then measured on the white background (3 measurements) and on the black background (3 measurements) using a Byk Gardner brand glossmeter of reference microTri-Gloss.

The haze index makes it possible to quantify the dulling of the nail varnish film. It was measured according to the following protocol:

A coat with a wet thickness of 300 μm of the composition whose mean gloss it was sought to evaluate was spread onto a Leneta brand contrast card of reference Form 1A Penopac, using an automatic spreader. The coat covered the white background and the black background of the card. The composition was left to dry for 24 hours on a bench thermostatically maintained at 30° C., and the haze index was then measured on the black background using a Byk Gardner brand glossmeter/hazemeter of reference Haze Gloss. This measurement was repeated five times, and the haze index value obtained corresponds to the calculated mean of the five measured values.

The following results were obtained:

| | Inventive Composition 1 | Comparative Composition 1 |
|---|---|---|
| Gloss (20°) | 76.1 | 66.8 |
| Haze | 132 | 188 |

### Example 2

### Nail Varnish

Two nail varnishes having the following compositions (wt %) were prepared:

| | Inventive | Comparative |
|---|---|---|
| | Composition 2 | Composition 2 |
| Nitrocellulose containing 30% | 11.34 | 12.81 |
| isopropyl alcohol (viscosity: | | |
| E22-1/2 S) | | |
| Pure isopropyl alcohol | 3.70 | 3.70 |
| Nitrocellulose containing 30% | 1.40 | 1.66 |
| isopropyl alcohol (Azur E80 | | |
| from Bergerac) | | |
| Hydrogenated ketone/formalde- | 5.00 | 0 |
| hyde resin ⁽¹⁾ | | |
| Ethyl tosylamide resin | 4.77 | 4.77 |
| Glycerophthalic alkyd resin | 16.07 | 16.07 |
| esterified with branched fatty | | |
| acids, at 70% in ethyl acetate | | |
| (Beckosol ODE 230 70E from | | |
| Dainippon Ink and Chemicals) | | |
| Hectorite modified with stearyl- | 1.28 | 1.28 |
| benzyl-dimethylammonium | | |
| (Bentone 27 V from Elementis) | | |
| Ferric blue pigment | 0.0003 | 0.0003 |
| Brown iron oxide coated with | 0.03 | 0.03 |
| polyethylene oxide | | |
| Titanium oxide coated with | 0.54 | 0.54 |
| polyethylene oxide | | |
| Yellow lake sold under the name | 0.02 | 0.02 |
| D&C Yel 5 AL Lake C 69-4424 by | | |
| the company Sun | | |
| Ethyl acetate | 21.03 | 23.24 |
| Tributyl acetyl citrate | 2.06 | 2.06 |
| Butyl acetate | 32.71 | 33.76 |
| Citric acid monohydrate | 0.05 | 0.05 |

⁽¹⁾ sold under the name Synthetic Resin SK by the company Degussa.

The gloss and the haze index of each varnish were measured according to the protocol described in Example 1.

The following results were obtained:

| | Inventive Composition 2 | Comparative Composition 2 |
|---|---|---|
| Gloss (20°) | 78 | 61.6 |
| Haze | 140 | 191 |

### Example 3

### Nail Varnish

Two nail varnishes having the following compositions (weight %) were prepared:

| | Inventive | Comparative |
|---|---|---|
| | Composition 3 | Composition 3 |
| Nitrocellulose containing 30% | 8.40 | 10.48 |
| isopropyl alcohol (viscosity: | | |
| E22-1/2 S) | | |
| Pure isopropyl alcohol | 4.74 | 4.02 |
| Nitrocellulose containing 30% | 1.17 | 2.56 |
| isopropyl alcohol (Azur E80 | | |
| from Bergerac) | | |
| Methyl methacrylate/butyl | 1.00 | 1.00 |
| methacrylate copolymer | | |
| Hydrogenated ketone/formal- | 5.00 | 0 |
| dehyde resin ⁽¹⁾ | | |
| Ethyl tosylamide resin | 2.04 | 2.04 |
| Glycerophthalic alkyd resin | 6.87 | 6.87 |
| esterified with branched fatty | | |
| acids, at 70% in ethyl acetate | | |
| (Beckosol ODE 230 70E from | | |
| Dainippon Ink and Chemicals) | | |
| Hectorite modified with stearyl- | 1.27 | 1.27 |
| benzyl-dimethylammonium | | |
| (Bentone 27 V from Elementis) | | |
| Red lake sold under the name D&C | 0.21 | 0.21 |
| Red 7 CA Lake C 19-011 by the | | |
| company Sun | | |
| Titanium oxide coated with | 0.02 | 0.02 |
| polyethylene oxide | | |
| Bordeaux lake sold under the name | 0.56 | 0.56 |
| D&C Red 34 CA Lake C 24-012 by the | | |
| company Sun | | |
| Ethyl acetate | 33.14 | 31.09 |
| Tributyl acetyl citrate | 5.47 | 5.47 |
| Butyl acetate | 30.06 | 34.36 |
| Citric acid monohydrate | 0.05 | 0.05 |

⁽¹⁾ sold under the name Synthetic Resin SK by the company Degussa.

The gloss and the haze index of each varnish were measured according to the protocol described in Example 1.

The following results were obtained:

| | Inventive Composition 3 | Comparative Composition 3 |
|---|---|---|
| Gloss (20°) | 64.5 | 48.6 |
| Haze | 187 | 249 |

What is claimed is:

1. A nail varnish comprising an organic solvent medium, at least one ketone/aldehyde resin, and at least one gelling agent.

2. The nail varnish of claim 1, wherein the organic solvent medium comprises at least one organic solvent chosen from short-chain esters comprising from 3 to 8 carbon atoms.

3. The nail varnish of claim 1, wherein the organic solvent medium is present in the composition in an amount ranging from 10% to 95% by weight relative to the total weight of the composition.

4. The nail varnish of claim 1, wherein the at least one ketone/aldehyde resin is a product of polycondensation of a mixture of at least one ketone and of at least one aldehyde.

5. The nail varnish of claim 4, wherein the at least one ketone is chosen from aliphatic ketones comprising from 1 to 20 carbon atoms, cycloaliphatic ketones comprising from 5 to 20 carbon atoms, and mixtures thereof.

6. The nail varnish of claim 4, wherein the at least one ketone is a cycloaliphatic ketone chosen from acetone, acetophenone, methyl ethyl ketone, 2-heptanone, methyl isobutyl ketone, cyclopentanone, cyclododecanone, mixtures of 2,2,4- and 2,4,4-trimethyl cyclohexanone, cyclohexanone, mixtures of cycloheptanone and of cyclooctanone, and cyclohexanones substituted with at least one alkyl group comprising from 1 to 8 carbon atoms, and mixtures thereof.

7. The nail varnish of claim 6, wherein the at least one ketone is acetophenone.

8. The nail varnish of claim 4, wherein the at least one aldehyde is chosen from linear aldehydes comprising from 1 to 20 carbon atoms, branched aldehydes comprising from 5 to 20 carbon atoms, and mixtures thereof.

9. The nail varnish of claim 8, wherein the at least one aldehyde is chosen from formaldehyde, acetaldehyde, n-butyraldehyde, iso-butyraldehyde, valeraldehyde, and dodecanal.

10. The nail varnish of claim 9, wherein the at least one aldehyde is formaldehyde.

11. The nail varnish of claim 1, wherein the at least one ketone/aldehyde resin is an acetophenone/formaldehyde resin.

12. The nail varnish of claim 1, wherein the at least one ketone/aldehyde resin is a optionally modified hydrogenated acetophenone/formaldehyde resin.

13. The nail varnish of claim 12, wherein the hydrogenated acetophenone/formaldehyde resin is a modified hydrogenated acetophenone/formaldehyde resin obtained by reacting a hydrogenated acetophenone/formaldehyde resin with an isocyanate.

14. The nail varnish according to claim 13, wherein the isocyanate is a diisocyanate chosen from linear and branched C₁-C₁₂ alkyl diisocyanates, C₄-C₂₀ cycloalkyl diisocyanates, and C₆-C₂₀ aryl diisocyanates.

15. The nail varnish of claim 13, wherein the modified hydrogenated acetophenone/formaldehyde resin is obtained by reacting a hydrogenated acetophenone/formaldehyde resin with isophorone diisocyanate (IPDI).

16. The nail varnish of claim 1, wherein the at least one ketone/aldehyde resin is present in an amount ranging from 0.01% to 20% by weight of active material relative to the total weight of the composition.

17. The nail varnish of claim 1, further comprising at least one film-forming polymer.

18. The nail varnish of claim 17, wherein the at least one film-forming polymer is present in an amount ranging from 0.1% to 60% by weight relative to the total weight of the composition.

19. The nail varnish of claim 1, further comprising at least one auxiliary film-forming agent.

20. The nail varnish of claim 1, wherein the at least one gelling agent comprises at least one organophilic clay.

21. The nail varnish of claim 20, wherein the at least one organophilic clay is chosen from modified hectorites.

22. The nail varnish of claim 20, wherein the at least one organophilic clay is present in an amount of greater than or equal to 0.5% by weight relative to the total weight of the composition.

23. The nail varnish according to claim 1, further comprising at least one dyestuff, present in an amount ranging from 0.01% to 50% by weight relative to the total weight of the composition.

24. A cosmetic process for making up and/or for the non-therapeutic care of the nails, comprising applying to the nails at least one coat of a nail varnish comprising an organic solvent medium, at least one ketone/aldehyde resin, and at least one gelling agent.

25. A process for obtaining a glossy film on the nails, comprising applying to the nails at least one coat of a nail varnish comprising an organic solvent medium, at least one ketone/aldehyde resin, and at least one gelling agent.
